(19) United States
(12) Patent Application Publication
(10) Pub. No.: US 2003/0031711 A1
(43) Pub. Date: Feb. 13, 2003

(54) Method of treating gastroesophageal reflux disease and nocturnal acid breakthrough

(76) Inventors: John W. Fara, Saratoga, CA (US); Bret Berner, El Granada, CA (US); Verne E. Cowles, Dublin, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/152,914

(22) Filed: May 20, 2002

Related U.S. Application Data
(60) Provisional application No. 60/294,551, filed on May 29, 2001.

(51) Int. Cl. A61K 9/46

(52) U.S. Cl. 424/466

(57) ABSTRACT

A method of concurrent treatment for gastroesophageal reflux disease and nocturnal acid breakthrough is described, which comprises the delivery of an GABA_{B} receptor agonist such as 4-amino-3-(4-chlorophenyl)butanoic acid, in the evening, in a gastric retained drug delivery system.

### CROSS REFERENCE TO RELATED APPLICATION

The present invention is related to and claims priority to U.S. Provisional Patent Application Serial No. 60/294,551, filed May 29, 2001, entitled “Method of treating gastroesophageal reflux disease and nocturnal acid breakthrough” which is incorporated herein by reference.

### TECHNICAL FIELD

The present invention relates to the use of GABA_{B} receptor agonists, and in particular baclofen (4-amino-3-(4-chlorophenyl)butanoic acid) for the concurrent treatment of gastroesophageal reflux disease and nocturnal acid breakthrough.

### BACKGROUND

Gastroesophageal reflux disease (“GERD”) may be caused by a variety of mechanisms, which include transient lower esophageal sphincter relaxations (“TLESRs”), decreased lower esophageal sphincter resting tone, impaired esophageal acid clearance, delayed gastric emptying, decreased salivation and impaired tissue resistance. GERD episodes typically occur during the early daytime hours, but some GERD sufferers also experience reflux during the night, even when being treated with proton pump inhibitors. These nighttime episodes of reflux are referred to as nocturnal acid breakthrough (“NAB”). For patients taking proton pump inhibitors, NAB is defined as a nocturnal gastric pH less than 4 for greater than 1 hour.

There are numerous treatments available for GERD. Martin, U.S. Pat. No. 5,036,057 describes treating GERD (heartburn) with a local anaesthetic in a dosage form designed to float on the gastrointestinal (“GI”) fluids contained in the stomach. Other treatments include administering proton pump inhibitors, histamine H2-receptor blockers and antacids such as described in Scott, et al., American Family Physician March 1999.

However, these remedies tend to be directed at alleviating the symptoms of GERD rather than treating the underlying causes. In addition, none of these address the often accompanying problem of NAB. Recent developments in the treatment of GERD include the administration of GABA_{B} receptor agonists. This is described in Andrews, et al., U.S. Pat. No. 6,117,908, which exemplifies the intravenous administration of 4-amino-3-(4-chlorophenyl) butanoic acid (“baclofen”). Baclofen, itself was described in 1969 in Keberle, et al., U.S. Pat. No. 3,471,548, and was first used as an agent to inhibit the central nervous system. Since then, baclofen has been extensively studied, both for its therapeutic applications and the various means by which the agent could be administered. For example, numerous studies have been conducted on the use of baclofen for the treatment of chronic hiccups, an affliction that often occurs in conjunction with gastroesophageal disease. See for example, Gueland, et al., European Respiratory Journal 8(2):235-237, 1995.

One of the problems encountered with administering baclofen is that the compound has a short half life and thus, is quickly eliminated. This becomes problematic when attempting to provide long-term relief such as is needed when developing a therapeutic regimen that will serve to treat both GERD, which tends to manifest itself during the waking hours, and NAB, which affects a patient throughout the night. Naturally, this problem can be addressed by giving multiple dosages. However, there are numerous disadvantages to this approach. For example, in order to treat NAB with conventional baclofen dosage forms, the patient must awaken in the middle of the night to take another dose. By requiring that several dosages be administered daily, the chances of missing a dose or duplicating a dose is increased. In addition, it is more difficult to maintain consistent plasma levels of the drug since there may be significant variances in the times that the patient take the dosages each day. For that reason, a once-daily or twice-daily dose regimen is preferred for the combined treatment of GERD and NAB.

Another problem encountered with adminstering baclofen is that absorption of baclofen into the blood stream occurs only in the upper gastrointestinal tract. Therefore, extended release versions of the most commonly used dosage forms such as tablets, capsules, and liquid formulations are not suitable for delivery of baclofen to treat GERD and NAB. However, there are several drug delivery systems that are suitable for use in the method of treatment of the invention as they are particularly tailored to be gastric-retained dosages, such as those described in Sinnreich, U.S. Pat. No. 4,996,058; Franz, et al., U.S. Pat. No. 5,232,704; Wong, et al., U.S. Pat. No. 6,120,803; Shell, et al., U.S. Pat. No. 5,972,389; and Shell, et al., WO 9855107.

These problems are addressed by the instant invention, which provides for the delivery of baclofen, alone or in combination with other therapeutic agents, by means of a gastric retained drug delivery system to treat GERD and NAB.

### SUMMARY OF THE INVENTION

One aspect of the invention relates to a method of concurrently treating gastroesophageal reflux disease and nocturnal acid breakthrough comprising administering a therapeutically effective amount of a GABA_{B} receptor agonist in the evening to a mammal in need of such treatment.

Another aspect of the invention pertains to a method of concurrently treating gastroesophageal reflux disease and nocturnal acid breakthrough comprising administering a therapeutically effective amount of 4-amino-3-(4-chlorophenyl) butanoic acid (“baclofen”), or a pharmaceutically acceptable salt or an optical isomer thereof in the evening to a mammal in need of such treatment.

Still yet another aspect of the invention relates to a method of concurrently treating gastroesophageal reflux disease and nocturnal acid breakthrough comprising administering a therapeutically effective amount of the R enantiomer of 4-amino-3-(4-chlorophenyl) butanoic acid in the evening to a mammal in need of such treatment.

Another aspect of the invention pertains to a method of concurrently treating gastroesophageal reflux disease and nocturnal acid breakthrough comprising administering a therapeutically effective amount a GABA_{B} receptor agonist in the evening to a mammal in need of such treatment, in combination with a therapeutic agent selected from the group consisting of proton pump inhibitors and histamine H2-receptor blockers.

### BRIEF DESCRIPTION OF DRAWINGS

FIG. 1 illustrates plasma concentration of Baclofen following administration of 20-mg Baclofen as Lioresal®, the commercially available immediate release product, or Baclofen, extended release, a gastric retentive tablet.

FIG. 2 illustrates plasma concentration of Baclofen following administration of 20 mg Baclofen as Lioresal®, the commercially available immediate release product or a Baclofen EGTS, a gastric retentive drug delivery formulation.

### DESCRIPTION OF THE INVENTION

It is very common to experience slight acid reflux, particularly after meals. In general, acid reflux irritates the esophageal walls, which induces peristaltic contraction of the esophageal smooth muscle. Depending upon the severity of the irritation and subsequent contraction to clear the refluxed acid, one may experience discomfort and even pain, which is commonly referred to as heartburn.

After a meal, the lower esophageal sphincter (“LES”) usually remains closed. However, when it relaxes at an inappropriate time, it allows acid and food particles to reflux into the esophagus. The process of secondary peristalsis returns most of the acid and food to the stomach and then the LES closes again. Any acid remaining in the esophagus is neutralized by saliva, and then is cleared into the stomach. Patients with GERD experience an increased number of transient LES relaxations and therefore, more frequent reflux episodes which increases the cumulative amount of time gastric acid spends in the esophagus. In addition, there are other factors that add to the increased esophageal acid exposure time that GERD patients experience, such as a decrease in the amplitude of secondary peristaltic waves which results in less effective esophageal acid clearance.

Eventually, GERD patients experience more than discomfort as the extent and severity of esophageal mucosal injury worsens. The associated pathological conditions include a variety of esophageal disorders such as erythema, isolated, confluent and circumferential erosions, deep ulcers, esophageal stricture and replacement of normal esophageal epithelium with abnormal (Barrett's) epithelium, which is a precancerous condition. Patients may also experience pain (odynophagia) or difficulty in swallowing (dysphagia); pulmonary symptoms such as chronic coughing, wheezing, asthma, aspiration pneumonia, and interstitial fibrosis; oral symptoms such as tooth enamel decay, gingivitis and halitosis; throat symptoms such as a soreness, laryngitis, hoarseness, and a globus sensation; and earache.

Most therapies have been directed to treating the more common daytime reflux episodes.

However, such treatments do not address reflux episodes that can occur during the evening hours or with nocturnal acid breakthrough (“NAB”). The instant invention is directed towards treating not only the underlying cause of GERD but also towards alleviation of reflux at nighttime and during NAB.

### Method of Treatment

The instant invention is a method of concurrently treating gastroesophageal reflux disease and nocturnal acid breakthrough comprising administering to a mammal in need of such treatment a therapeutically effective amount of a GABA_{B} receptor agonist.

As used herein, the term “treating” covers treating the disease of GERD and NAB in a mammal, particularly a human, and includes:

(i) preventing the disease from occurring in a subject which may be predisposed to the disease but has not yet been diagnosed as having it;

(ii) inhibiting the disease, i.e. arresting its development; or

(iii) relieving the disease, i.e. causing regression of the disease.

In another embodiment of the invention, the method comprises administering a therapeutically effective amount of 4-amino-3-(4-chlorophenyl)butanoic acid (“baclofen”), or a pharmaceutically acceptable salt or an optical isomer thereof. In still another embodiment of the invention the R enantiomer of 4-amino-3-(4-chlorophenyl) butanoic acid is administered.

The invention also contemplates administering one or more additional therapeutic agents with the GABA_{B} receptor agonist treatment. Such additional therapeutic agents are selected from the group consisting of proton pump inhibitors and histamine H2-receptor blockers.

### GABA_{B} Receptor Agonist

There are numerous GABA_{B} receptor agonists suitable for use in the methods of the invention. These include by way of illustration and not limitation, γ-amino-β-(p-halophenyl)-butyric acids and their esters (Keberle, et al., U.S. Pat. No. 3,471,548), as well as the pharmaceutically acceptable salts or optical isomers thereof.

Of particular interest are the substituted aminopropyl acid derivatives described in Andrews, et al., U.S. Pat. No. 6,117,908. These include by way of illustration and not limitation: 4-aminobutanoic acid; 4-amino-3-(4-chlorophenyl) butanoic acid (baclofen); 4-amino-3-phenylbutanoic acid; 4-amino-3-hydroxybutanoic acid; 4-amino-3-(4-chlorophenyl)-3-hydroxyphenylbutanoic acid; 4-amino-3-(thien-2-yl) butanoic acid; 4-amino-3-(5-chlorothien-2-yl) butanoic acid; 4-amino-3-(5-bromothien-2-yl) butanoic acid; 4-amino-3-(5-methylthien-2-yl) butanoic acid; 4-amino-3-(2-imidazolyl) butanoic acid; 4-guanidino-3-(4-chlorophenyl) butanoic acid; 3-amino-2-(4-chlorophenyl)-1-nitropropane; (3-aminopropyl) phosphonous acid; (4-aminobut-2-yl) phosphonous acid; (3-amino-2-methylpropyl) phosphonous acid; (3-aminobutyl) phosphonous acid; (3-amino-2-(4-chlorophenyl)propyl) phosphonous acid; (3-amino-2-(4-chlorophenyl)-2-hydroxypropyl) phosphonous acid; (3-amino-2-(4-fluorophenyl)propyl) phosphonous acid; (3-amino-2-phenylpropyl) phosphonous acid; (3-amino-2-hydroxypropyl) phosphonous acid; (E)-(3-aminopropen-1-yl) phosphonous acid; (3-amino-2-cyclohexylpropyl) phosphonous acid; (3-amino-2-benzylpropyl) phosphonous acid; [3-amino-2-(4-methylphenyl)propyl] phosphonous acid; [3-amino-2-(4-trifluoromethylphenyl)propyl] phosphonous acid; [3-amino-2-(4-methoxyphenyl)propyl] phosphonous acid; [3-amino-2-(4-chlorophenyl)-2-hydroxypropyl] phosphonous acid; (3-aminopropyl) methylphosphinic acid; (3-amino-2-hydroxypropyl) methylphosphinic acid; (3-aminopropyl)(difluoromethyl) phosphinic acid; (4-aminobut-2-yl) methylphosphinic acid; (3-amino-1-hydroxypropyl)methylphosphinic acid; (3-amino-2-hydroxypropyl)(difluoromethyl) phosphinic acid; (E)-(3-aminopropen-1-yl) methylphosphinic acid; (3-amino-2-oxo-propyl) methyl phosphinic acid; (3-aminopropyl) hydroxymethylphosphinic acid; (5-aminopent-3-yl) methylphosphinic acid; (4-amino-1,1,1-trifluorobut-2-yl) methylphosphinic acid; (3-amino-2-(4-chlorophenyl)propyl) sulfinic acid and 3-aminopropylsulfinic acid.

A particularly useful GABA_{B} receptor agonist is the γ-amino-β-(p-halophenyl)-butyric acid referred to as 4-amino-3-(4-chlorophenyl) butanoic acid (“baclofen”).

### Additional Therapeutic Agents

The methods of the invention also contemplate the addition of one or more therapeutic agents with the GABA_{B} receptor agonist treatment. Such additional therapeutic agents are selected from the group consisting of proton pump inhibitors and histamine H2-receptor blockers.

Proton pump inhibitors act by inhibiting gastric acid secretion. Examples of proton pump inhibitors that can be used in the methods of the invention include, by way of illustration and not limitation, omeprazole, lansoprazole, pantoprazole, rabeprazole and esomeprazole.

Histamine H2-receptor blockers are administered to both prevent and relieve reflux symptoms such as heartburn, acid indigestion and sour stomach as well as being used to treat duodenal ulcers and prevent their return. Histamine H2-receptor blockers act by inhibiting histamine stimulation of the gastric parietal cell and thereby suppress gastric acid secretion. Examples of histamine H2-receptor blockers that can be used in the methods of the invention include, by way of illustration and not limitation, cimetidine and cimetidine HCl, famotidine, nizatidine, ranitidine and ranitidine HCl, and other suitable salts.

### Forms of GABA_{B} Receptor Agonist and Additional Therapeutic Agents

Pharmaceutically acceptable salts of the agonist or the additional therapeutic agent(s) can also be used in the methods of the invention as long as the salt form retains the biological effectiveness and properties of the agonist or the additional therapeutic agent(s), and are not biologically or otherwise undesirable. Such pharmaceutically acceptable salts may be amphoteric and may be present in the form of internal salts. The agonist and other agents may form acid addition salts and salts with bases. Exemplary acids that can be used to form such salts include, by way of example and not limitation, mineral acids such as hydrochloric, hydrobromic, sulfuric or phosphoric acid or organic acids such as organic sulfonic acids and organic carboxylic acids. Salts formed with inorganic bases include, for example, the sodium, potassium, lithium, ammonium, calcium, and magnesium salts. Salts derived from organic bases include, for example, the salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethyl aminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, fumarate, maleate, succinate, acetate and oxalate.

Optical isomers can also be used in the methods of the invention. For example, the agonist baclofen, is a chiral compound due to the presence of an asymmetric carbon atom. Accordingly, baclofen may be administered in the form of mixtures of isomers (e.g., racemates), or in the form of pure isomers (e.g., enantiomers).

Accordingly, as used herein the terms “GABA_{B} receptor agonist” and “therapeutic agent” are intended to include the compounds themselves as well as their pharmaceutically acceptable salts and optical isomers.

### Dosage

In general, the term “therapeutically effective amount” refers to that amount which is sufficient to effect treatment, when administered to a mammal in need of such treatment. The therapeutically effective amount will vary depending on the subject being treated, the severity of the disease state and the manner of administration, and may be determined routinely by one of ordinary skill in the art.

In particular, for use in the treatment of gastroesophageal reflux disease and nocturnal acid breakthrough, GABA_{B} receptor agonists such as baclofen may be used at doses appropriate for other conditions for which other GABA_{B} receptor agonists have been administered. Typically, the method of the invention will involve administering the GABA_{B} receptor agonist on a daily basis for as long as the conditions (GERD and NAB) persist. An effective dosage is typically in the range of about 5-100 mg/dosage, typically about 10-80 mg/dosage, more typically about 20-60 mg/dosage.

If a proton pump inhibitor is also included in the method of the invention, the dosage is typically in the range of about 15-100 mg/dosage, typically about 15-80 mg/dosage, more typically about 15-60 mg/dosage.

If a histamine H2-receptor blocker is also included in the method of the invention, the dosage is typically in the range of about 20-800 mg/dosage, typically about 20-500 mg/dosage, more typically about 20-400 mg/dosage.

### Dosage Regimen

There are several dosage regimens that are suitable for use with the methods of the invention.

In one embodiment of the invention, a GABA_{B} receptor agonist is administered in the evening, for example, with the evening meal or near bedtime.

In another aspect of the invention, the method of administering a GABA_{B} receptor agonist in the evening further includes administering an additional therapeutic agent simultaneously with the administration of the GABA_{B} receptor agonist, said agent being selected from the group consisting of proton pump inhibitors, histamine H2-receptor blockers and combinations thereof. As used herein the term “simultaneous” is intended to mean administration of the agonist and additional agent at approximately the same time, i.e., in the evening and therefore includes administration together and administration of the agonist and agent within a few hours of each other.

In another aspect of the invention, the method of administering a GABA_{B} receptor agonist in the evening further includes administering an additional therapeutic agent in the daytime, where the additional agent is selected from the group consisting of GABA_{B} receptor agonists, proton pump inhibitors, histamine H2-receptor blockers and combinations thereof. Typically this additional agent would be administered in the morning, for example with breakfast.

In yet another embodiment of the invention, the method of administering a GABA_{B} receptor agonist in the evening further includes administering an additional therapeutic agent simultaneously with the administration of the GABA_{B} receptor agonist and administering an additional therapeutic agent in the daytime.

One exemplary therapeutic regimen is administering a smaller dose of a GABA_{B} receptor agonist in the morning, followed by a larger dose of an GABA_{B} receptor agonist in the evening, where the smaller dosage in the morning also serves to minimize any sedation effects.

Another exemplary therapeutic regimen is administering a proton pump inhibitor or histamine H2-receptor blocker in the morning, followed by administering an GABA_{B} receptor agonist in the evening, where the evening dosage optionally includes a proton pump inhibitor or histamine H2-receptor blocker.

### Dosage Form-Evening Dose

There are several drug delivery systems that are suitable for use in delivering the evening dosage form of the GABA_{B} receptor agonist as they are particularly tailored to be gastric-retained dosages, such as the swellable bilayer described by Franz, et al., U.S. Pat. No. 5,232,704; the multi-layer tablet with a band described by Wong, et al., U.S. Pat. No. 6,120,803; the membrane sac and gas generating agent described in Sinnreich, U.S. Pat. No. 4,996,058; the swellable, hydrophilic polymer system described in Shell, et al., U.S. Pat. No. 5,972,389 and Shell, et al., WO 9855107; and the buccal system described in Khanna, et al., U.S. Pat. No. 5,091,184, all of which are incorporated herein by reference. Of particular interest are gastric retained dosage forms that contain hydrophilic polymers that swell to a size such that the dosage form is retained in the fed mode.

A typical dosage form would provide for a drug delivery profile such that the agonist is delivered for a period of at least 6 hours. In order to provide for sustained delivery, it is preferable that at least 40 wt % of the agonist is retained in the dosage form after 1 hour, i.e., no more than 60 wt % of the drug is administered in the first hour. In addition, it may be desired to utilize a dosage form that provides for substantially all of the agonist to be delivered over the intended duration, which is typically about 6-24 hours, where substantially all is taken to mean at least about 85 wt % of the agonist is administered.

In one embodiment of the invention, the evening dosage form of the GABA_{B} receptor agonist is a film coated dosage form or a capsule dosage form that allows for the extended release of the GABA_{B} receptor agonist in the stomach and comprises: (a) at least one component that expands on contact with gastric juice and contains an agent capable of releasing carbon dioxide or nitrogen, a GABA_{B} receptor agonist; (b) at least one hydrophilic membrane in the form of a sachet which contains component (a), expands by inflation, floats on the aqueous phase in the stomach and is permeable to gastric juice and; (c) a film coating or capsule form which contains components (a) and (b) and which disintegrates without delay in the stomach under the action of gastric juice. Component (a) may also contain a pharmaceutically acceptable hydrophilic swelling agent such as lower alkyl ethers of cellulose, starches, water-soluble aliphatic or cyclic poly-N-vinylamides, polyvinyl alcohols, polyacrylates, polymethacrylates, polyethylene glycols and mixtures thereof, as well as other materials used in the manufacture of pharmaceutical dosage forms. Further details regarding an example of this type of dosage form can be found in Sinnreich, U.S. Pat. No. 4,996,058.

In another embodiment of the invention, the evening dosage form of the GABA_{B} receptor agonist is an extended release oral drug dosage form for releasing the GABA_{B} receptor agonist into the stomach, duodenum and small intestine of a patient, and comprises: a plurality of solid particles consisting of the GABA_{B} receptor agonist dispersed within a polymer that (i) swells unrestrained dimensionally by imbibing water from gastric fluid to increase the size of the particles to promote gastric retention in the stomach of the patient in which the fed mode has been induced; (ii) gradually the drug diffuses or the polymer erodes over a time period of hours, where the diffusion or erosion commences upon contact with the gastric fluid; and (iii) releases the agonist to the stomach, duodenum and small intestine of the patient, as a result of the diffusion or polymeric erosion at a rate corresponding to the time period. Exemplary polymers include polyethylene oxides, alkyl substituted cellulose materials and combinations thereof, for example, high molecular weight polyethylene oxides and high molecular weight or viscosity hydroxypropylmethylcellulose materials. Further details regarding an example of this type of dosage form can be found in Shell, et al., U.S. Pat. No. 5,972,389 and Shell, et al., WO 9855107.

In yet another embodiment, a bi-layer tablet releases the GABA_{B} receptor agonist to the upper gastrointestinal tract from an active containing layer, while the other layer is a buoyant or floating layer. Details of this dosage may be found in Franz, et al., U.S. Pat. No. 5,232,704. The dosage form of the present invention may be surrounded by a band of insoluble material as described by Wong, et al., U.S. Pat. No. 6,120,803.

In still another embodiment of the invention, the evening dosage form of the GABA_{B} receptor agonist is a pharmaceutical composition in the form of an adhesive tablet, and comprises a hydrophobic tablet core, the top surface of which adheres to the receptor surface of the oral mucosa, and which consists of the GABA_{B} receptor agonist. The tablet may contain excipients such as a swellable vinyl polymer, a galactomannan, a wax, a glyceride, a completely hydrogenated glyceride and a partially hydrogenated glyceride. In addition, the tablet may have a hydrophobic coating which covers the tablet core with the exception of the surface provided for the release of the GABA_{B} receptor agonist. Further details regarding this dosage form can be found in Khanna, et al., U.S. Pat. No. 5,091,184.

In another embodiment of the invention, the GABA_{B} receptor agonist is delivered systemically through the skin throughout the day and night as a transdermal patch, as described in Mazzenga, et al., U.S. Pat. No. 5,073,539.

For those embodiments of the invention that include further administering a proton pump inhibitor or histamine H2-receptor blocker simultaneously with the GABA_{B} receptor agonist, the proton pump inhibitor or histamine H2-receptor blocker can either be administered in a dosage form that includes the GABA_{B} receptor agonist or can be administered in a dosage form that is separate from the GABA_{B} receptor agonist. Exemplary dosage forms are described below.

### Dosage Form-Daytime Dose

For those embodiments of the invention that include further administering one or more additional therapeutic agents in the daytime, typically in the morning such as with breakfast, the daytime dosage can be any suitable formulation as are well known in the art.

When the method of the invention includes administering a GABA_{B} receptor agonist, proton pump inhibitor or histamine H2-receptor blocker in the morning, with the GABA_{B} receptor agonist being delivered in the evening, then there are numerous commercially available dosage forms that can be administered. In addition, other formulations can be readily designed based upon knowledge in the art, and include the gastric-retained delivery systems described above.

Typical dosage forms of the proton pump inhibitor suitable for use in the invention include capsules and tablets. One of skill in the art can readily prepare one of these exemplary formulations or the proton pump inhibitor can be administered by means of one of the numerous commercially available products, which include, for example, Prilosec® (omeprazole, AstraZenca), Prevacid® (lansoprazole, TAP Pharmaceutical Products, Inc.), Protonix® (pantoprazole, Wyeth-Ayerst Laboratories) and Aciphex® (rabeprazole, Eisan, Inc.).

Typical dosage forms of the histamine H2-receptor blocker suitable for use in the invention include syrups, solutions, suspensions, tablets (including chewable and oral disintegrating tablets), capsules, and effervescent formulations of granules or tablets. One of skill in the art can readily prepare one of these exemplary formulations or the histamine H2-receptor blocker can be administered by means of one of the numerous commercially available products, which include, for example, Tagamet® (cimetidine, GlaxoSmithKline), Pepcid® (famotidine, Merck & Co.), Axid® (nizatidine, Eli Lilly & Co.) and Zantac® (ranitidine, Pfizer).

Although specific examples of suitable proton pump inhibitor and histamine H2-receptor blocker formulations are described above, it is understood that the invention is not limited to those examples as there are numerous other formulations that can be used to deliver the morning dosage of the additional GABA_{B} receptor agonist, proton pump inhibitor or histamine H2-receptor blocker.

Typically, dosage forms contain the active agent (GABA_{B} receptor agonist, proton pump inhibitor or histamine H2-receptor blocker) in combination with one or more pharmaceutically acceptable ingredients. The carrier may be in the form of a solid, semi-solid or liquid diluent, or a capsule. Usually the amount of active agent is about 0.1-95 wt %, more typically about 1-50 wt %. Actual methods of preparing such dosage forms are known, or will be apparent, to those skilled in this art; for example, see Remington's Pharmaceutical Sciences, Mack Publishing Company, Easton, Pa., 18th Edition, 1990. The dosage form to be administered will, in any event, contain a quantity of the additional therapeutic agent(s) in an amount effective to alleviate the symptoms of the subject being treated.

In the preparation of pharmaceutical formulations containing the additional therapeutic agent in the form of dosage units for oral administration the agent may be mixed with solid, powdered ingredients, such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or pressed into tablets such as chewable and oral disintegrating tablets.

Soft gelatin capsules may be prepared by mixing the active agent and vegetable oil, fat, or other suitable vehicle. Hard gelatin capsules may contain granules of the active agent, alone or in combination with solid powdered ingredients such as lactose, saccharose, sorbitol, mannitol, potato starch, corn starch, amylopectin, cellulose derivatives or gelatin.

Liquid preparations for oral administration may be prepared in the form of syrups or suspensions, e.g. solutions or suspensions containing about 0.2-20 wt % of the active agent and the remainder consisting of sugar or sugar alcohols and a mixture of ethanol, water, glycerol, propylene glycol and polyethylene glycol. If desired, such liquid preparations may contain coloring agents, flavoring agents, saccharin and carboxymethyl cellulose or other thickening agents. Liquid preparations for oral administration may also be prepared in the form of a dry powder to be reconstituted with a suitable solvent prior to use.

The general methods of the invention are best understood with reference to the following examples which are intended to enable those skilled in the art to more clearly understand and to practice the present invention. These examples are not intended, nor are they to be construed, as limiting the scope of the invention, but are merely illustrative and representative thereof.

### EXAMPLE 1

Tablets weighing 715 mg, were prepared with 20 mg of USP baclofen containing 367.8 mg of microcrystalline cellulose, 122.56 mg lactose, 23.57 mg hydroxypropylmethylcellulose, 171.6 mg of polyethylene oxide and 3.58 mg of magnesium stearate. 5.89 mg was residual water from processing. 91% of the baclofen released into 0.1 N HCl in 10 hours.

### EXAMPLE 2

Tablets weighing 715 mg, were prepared with 20 mg of USP baclofen containing 196.2 mg of microcrystalline cellulose, 122.56 mg lactose, 23.57 mg hydroxypropylmethylcellulose, 343.2 mg of polyethylene oxide and 3.58 mg of magnesium stearate. 5.89 mg was residual water from processing. 82.3% of the baclofen released into 0.1 N HCl in 10 hours.

### EXAMPLE 3

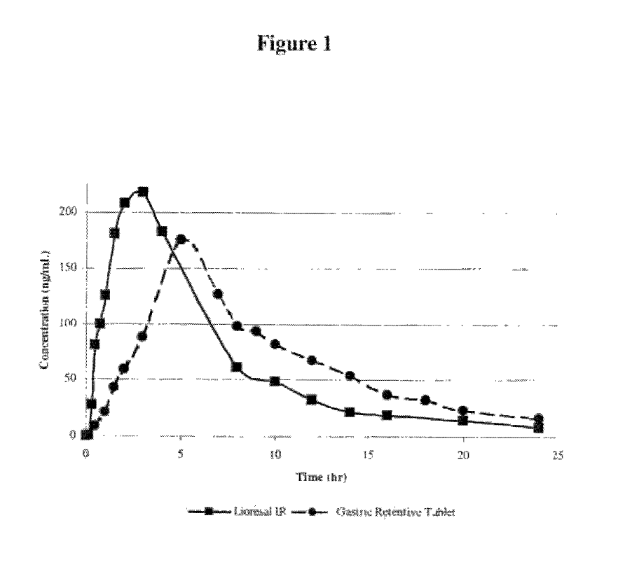

Tablets from Example 2 were made with an Amberlite® ion exchange resin containing 1 MBq of¹¹¹ Indium. The tablets were administered to 4 healthy volunteers after a low fat breakfast and visualized by gamma scintigraphy. The mean residence time in the upper gastrointestinal tract, i.e., stomach and small intestine, was 8.7±3.7 hours. Blood samples were taken at specified intervals and analyzed for Baclofen concentration in the plasma and compared to plasma concentration in the same subjects after administration of the immediate release baclofen tablet, Lioresal® 20-mg. FIG. 1 illustrates plasma concentration of Baclofen following administration of 20-mg Baclofen as Lioresal®, the commercially available immediate release product, or Baclofen, extended release, a gastric retentive tablet. FIG. 1 and Table 1 demonstrate the expected extended release attributes with a lower maximum concentration and later time of maximum concentration as compared to the immediate release product.

The pharmacokinetic parameters for this study are provided in Table 1.

TABLE 1
Pharmacokinetic Parameters

| | Lioresal | Baclofen |
|---|---|---|
| | (immediate release) | extended release |
| AUC (ng/ml*hr) | 1533 ± 310 | 1551 ± 277 |
| Cₘₐₓ (ng/ml) | 255 ± 63 | 176 ± 57 |
| tₘₐₓ (hour) | 1.8 ± 1.0 | 5 ± 0 |

### EXAMPLE 4

The Endo Gastric Therapeutic Systems described in U.S. Pat. 4,996,058 Sinnreich et al, and hereby incorporated by reference, were manufactured as follows:

An EGTS PolyVinylAcetate laminate pouch containing, 20 mg USP Baclofen compressed with 482.7 mg of sodium bicarbonate, 85.26 mg Myrj 52FL (polyethylene glycol (40) monostearate) together with 50 mg compressed citric acid were encapsulated in a gelatin capsule to give formulation 1.

An EGTS PolyVinylAcetate laminate pouch containing 20 mg USP Baclofen compressed with 482.7 mg of sodium bicarbonate, 85.26 mg Myrj 52FL (polyethylene glycol (40) monostearate) was encapsulated in gelatin capsule to give formulation 2.

### EXAMPLE 5

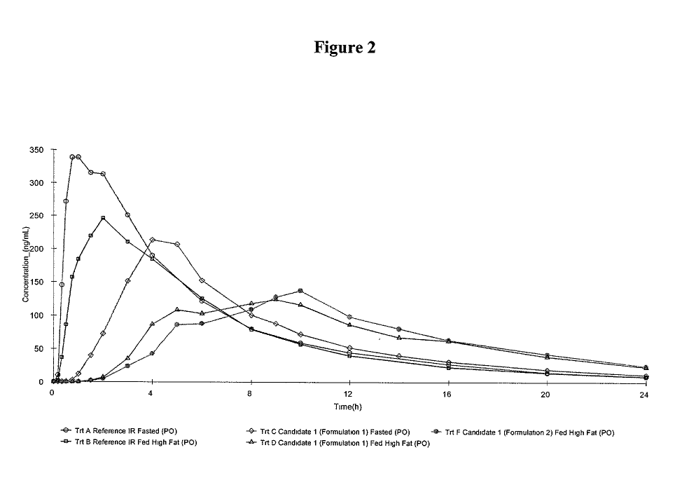

Formulations 1 and 2 from example 4 were administered to normal healthy volunteers (n=12) in a cross over, pharmacoscintigraphy study. Each subject was dosed with 20 mg Baclofen as Lioresal or EGTS formulation 1 or EGTS formulation 2. The Lioresal® and EGTS formulation 1 were administered fasted and after a high fat breakfast. The EGTS formulation 2 was administered after a high fat breakfast. Blood samples were taken at specified intervals and analyzed for Baclofen. The gastric residence of the Baclofen EGTS formulations was visualized by gamma scintigraphy. The mean residence times in the stomach were: 8.3+/−8.8,20+/−0 and 19.3+/−3.3 hours for formulation 1 fasted and fed, and for formulation 2 fed, respectively. The pharmacokinetic parameters for this study are provided in Table 2. FIG. 2 illustrates plasma concentration of Baclofen following administration of 20 mg baclofen as Lioresal®, the commercially available immediate release product or a Baclofen EGTS, a gastric retentive drug delivery formulation. FIG. 2 and Table 2 demonstrate the expected extended release attributes with a lower maximum concentration and later time of maximum concentration as compared to the immediate release product.

TABLE 2
Mean Pharmacokinetic parameters

| | Trt A | Trt B | Trt C | Trt D | Trt E |
|---|---|---|---|---|---|
| | Reference IR | Reference IR | (Formulation | (Formulation 1) | (Formulation 2) |
| PK | Fasted | Fed High Fat | 1) Fasted | Fed High Fat | Fed High Fat |
| Parameters | n = 14 | n = 14 | n = 13 | n = 13 | n = 12 |
| F | — | — | 80.4 ± 26.0 | 97.9 ± 19.0 | 99.9 ± 19.4 |
| (%) | | | | | |
| CV % | — | — | 32.4 | 19.4 | 19.4 |
| Relative to | | | | | |
| Lioresal in the | | | | | |
| same state | | | | | |
| AUClast | 2061.2 ± 572.1 | 1726.5 ± 273.8 | 1600.1 ± 710.6 | 1558.6 ± 319.0 | 1543.8 ± 347.3 |
| (ng/mL.h) | | | | | |
| CV % | 27.8 | 15.9 | 44.4 | 20.5 | 22.5 |
| Cmax | 385.7 ± 85.9 | 275.0 ± 53.6 | 234.8 ± 128.7 | 158.8 ± 62.0 | 157.3 ± 44.4 |
| (ng/mL) | | | | | |
| CV % | 22.3 | 19.5 | 54.8 | 39.0 | 28.2 |
| Tmax | 1.1 ± 0.5 | 1.6 ± 0.8 | 4.3 ± 0.5 | 8.31 ± 1.9 | 8.5 ± 1.8 |
| (h) | | | | | |
| CV % | 45.2 | 50.0 | 11.2 | 22.7 | 20.6 |

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of concurrently treating gastroesophageal reflux disease and nocturnal acid breakthrough comprising administering a therapeutically effective amount of a GABA_{B} receptor agonist in the evening to a mammal in need of such treatment.

2. The method of claim 1 wherein the agonist is administered with the evening meal or near bedtime.

3. The method of claim 1 wherein the agonist is administered in a gastric retained drug delivery system.

4. The method of claim 3 wherein said gastric retained drug delivery system is an extended release oral drug dosage form for releasing the agonist into the stomach, duodenum and small intestine of the mammal.

5. The method of claim 4 wherein the agonist is administered from the dosage form for a period of at least 6 hours and at least 40 wt % of the agonist is retained after 1 hour.

6. The method of claim 5 wherein the dosage form provides for substantially all of the agonist to be delivered over a period of about 6-24 hours.

7. The method of claim 5 wherein the dosage form contains a hydrophilic polymer that swells to a size such that the dosage form is retained in the fed mode.

8. The method of claim 7 wherein the polymer is selected from the group consisting of polyethylene oxides, alkyl substituted cellulose materials, and combinations thereof.

9. The method of claim 5 wherein the dosage form further comprises a gas generating agent.

10. The method of claim 9 wherein the agonist is contained in a membrane sachet with the gas generating agent.

11. The method of claim 3 wherein said gastric retained drug delivery system is an adhesive tablet.

12. The method of claim 1 wherein said dosage is about 5-100 mg.

13. The method of claim 12 wherein said dosage is about 10-80 mg.

14. The method of claim 13 wherein said dosage is about 20-60 mg.

15. The method of claim 1 which further comprises administering a therapeutic agent selected from the group consisting of proton pump inhibitors, histamine H2-receptor blockers and combinations thereof.

16. The method of claim 15 wherein the therapeutic agent is administered in the evening.

17. The method of claim 15 wherein the therapeutic agent is administered in the daytime.

18. The method of claim 1 which further comprises administering a GABA_{B} receptor agonist in the daytime.

19. The method of claim 18 which further comprises administering a therapeutic agent selected from the group consisting of proton pump inhibitors, histamine H2-receptor blockers and combinations thereof.

20. A method of concurrently treating gastroesophageal reflux disease and nocturnal acid breakthrough comprising administering a therapeutically effective amount of 4-amino-3-(4-chlorophenyl)butanoic acid, or a pharmaceutically acceptable salt or an optical isomer thereof in the evening to a mammal in need of such treatment.

21. The method of claim 20 wherein 4-amino-3-(4-chlorophenyl)butanoic acid is administered with the evening meal or near bedtime.

22. The method of claim 20 wherein 4-amino-3-(4-chlorophenyl)butanoic acid is administered in a gastric retained drug delivery system.

23. The method of claim 22 wherein the gastric retained drug delivery system is a film coated dosage form or a capsule dosage form that allows for the extended release of 4-amino-3-(4-chlorophenyl)butanoic acid in the stomach.

24. The method of claim 22 wherein said gastric retained drug delivery system is an extended release oral drug dosage form for releasing 4-amino-3-(4-chlorophenyl)butanoic acid into the stomach, duodenum and small intestine of the mammal.

25. The method of claim 22 wherein said gastric retained drug delivery system is an adhesive tablet.

26. The method of claim 20 wherein said dosage is about 5-100 mg.

27. The method of claim 26 wherein said dosage is about 10-80 mg.

28. The method of claim 27 wherein said dosage is about 20-60 mg.

29. The method of claim 20 which further comprises administering a therapeutic agent selected from the group consisting of proton pump inhibitors, histamine H2-receptor blockers and combinations thereof.

30. The method of claim 29 wherein the therapeutic agent is administered in the evening.

31. The method of claim 29 wherein the therapeutic agent is administered in the daytime.

32. The method of claim 20 which further comprises administering a GABA_{B} receptor agonist in the daytime.

33. The method of claim 32 wherein the GABA_{B} receptor agonist is 4-amino-3-(4-chlorophenyl)butanoic acid.

34. The method of claim 32 which further comprises administering a therapeutic agent selected from the group consisting of proton pump inhibitors, histamine H2-receptor blockers and combinations thereof.

35. A method of concurrently treating gastroesophageal reflux disease and nocturnal acid breakthrough comprising administering a therapeutically effective amount of the R enantiomer of 4-amino-3-(4-chlorophenyl)butanoic acid in the evening to a mammal in need of such treatment.

36. The method of claim 35 wherein the R enantiomer is administered with the evening meal or near bedtime.

37. The method of claim 35 wherein the R enantiomer is administered in a gastric retained drug delivery system.

38. The method of claim 37 wherein the gastric retained drug delivery system is a film coated dosage form or a capsule dosage form that allows for the extended release of the R enantiomer in the stomach.

39. The method of claim 37 wherein said gastric retained drug delivery system is an extended release oral drug dosage form for releasing the R enantiomer into the stomach, duodenum and small intestine of the mammal.

40. The method of claim 37 wherein said gastric retained drug delivery system is an adhesive tablet.

41. The method of claim 35 wherein said dosage is about 5-100 mg.

42. The method of claim 41 wherein said dosage is about 10-80 mg.

43. The method of claim 42 wherein said dosage is about 20-60 mg.

44. The method of claim 35 which further comprises administering a therapeutic agent selected from the group consisting of proton pump inhibitors, histamine H2-receptor blockers and combinations thereof.

45. The method of claim 44 wherein the therapeutic agent is administered in the evening.

46. The method of claim 44 wherein the therapeutic agent is administered in the daytime.

47. The method of claim 35 which further comprises administering a GABA_{B} receptor agonist in the daytime.

48. The method of claim 47 wherein the GABA_{B} receptor agonist is the R enantiomer of 4-amino-3-(4-chlorophenyl)butanoic acid.

49. The method of claim 47 which further comprises administering a therapeutic agent selected from the group consisting of proton pump inhibitors, histamine H2-receptor blockers and combinations thereof.
